(19) United States
(12) Patent Application Publication
(10) Pub. No.: US 2024/0423892 A1
(43) Pub. Date: Dec. 26, 2024

(54) TRANSPARENT COMPOSITION COMPRISING POLYHYDROXY ACID

(71) Applicant: L'OREAL, Paris (FR)

(72) Inventor: Wei WANG, Kawasaki-shi (JP)

(21) Appl. No.: 18/685,385

(22) PCT Filed: Aug. 23, 2022
(86) PCT No.: PCT/JP2022/032303

§ 371 Date: Feb. 21, 2024

(30) Foreign Application Priority Data
Aug. 30, 2021 (JP) 2021-139849
Oct. 5, 2021 (FR) 2110524

(51) Int. Cl. A61K 8/49; A61K 8/365; A61K 8/67; A61K 8/86; A61Q 19/00

(52) U.S. Cl. CPC A61K 8/498; A61K 8/365; A61K 8/675; A61K 8/86; A61Q 19/00; A61K 2800/28

(57) ABSTRACT

A transparent composition contains: (a) at least one first compound selected from polyhydroxy acids and salts thereof; (b) at least one second compound selected from Vitamin B3 and derivatives thereof; (c) at least one alkyleneoxide derivative represented by a specific chemical formula; and (d) water. The amount of the (a) first compound(s) in the composition is 1% by weight or more, preferably 3% by weight or more, and more preferably 5% by weight or more, relative to the total weight of the composition. The composition is transparent, and can provide less stickiness and reduced skin discomfort such as reduced skin irritation, when the composition is applied onto the skin, although the composition includes a relatively large amount of polyhydroxy acid or salt thereof.

### TECHNICAL FIELD

The present invention relates to a composition, preferably a cosmetic composition, which comprises a relatively large amount of polyhydroxy acid.

### BACKGROUND ART

Peeling is a well-known means for improving the appearance of the surface of the skin, in particular for treating visible and/or tactile irregularities of the human skin, and for example to attenuate defects of pigmentation such as skin freckles or the marks due to acne or varicella, or to smooth irregularities in the texture of the skin, in particular wrinkles or minor wrinkles.

This peeling has the effect of removing part of the skin to be treated (epidermis and possibly upper layer of dermis) by chemical methods such as the application of compositions containing a peeling agent(s) stimulating the desquamation of the skin, for example alpha-hydroxy acids (AHAs) such as glycolic acid or beta-hydroxy acids (BHAs) such as salicylic acid, or else other active substances such as retinoic acid, resorcinol, trichloroacetic acid or phenol.

### DISCLOSURE OF INVENTION

Polyhydroxy acids (PHAs), as well as salts thereof, are also useful as peeling agents. In general, PHAs have a larger molecule size than AHAs. Therefore, PHAs do not tend to penetrate into the skin as deeply as AHAs when applied onto the skin. Thus, PHAs are in general less irritating and preferable for in particular sensitive skin.

However, it has been found that, if a relatively large amount of PHAs is used in a composition, the composition becomes sticky and causes skin discomfort such as skin irritation when the composition is applied onto the skin. Adding a substantial amount of oil to the composition may reduce the stickiness. However, the composition including a substantial amount of oil becomes not transparent. Transparency is sometimes important for consumer products such as cosmetics including skin peeling compositions.

An objective of the present invention is to provide a transparent composition which can provide less stickiness and causes reduced skin discomfort such as reduced skin irritation, when the composition is applied onto the skin, even if the composition includes a relatively large amount of polyhydroxy acid or salt thereof.

The above objective of the present invention can be achieved by a transparent composition comprising:
(a) at least one first compound selected from polyhydroxy acids and salts thereof;
(b) at least one second compound selected from Vitamin B3 and derivatives thereof;
(c) at least one alkyleneoxide derivative represented by the following chemical formula (I):

Z—{O(AO)ₗ(EO)ₘ-(BO)ₙH}ₐ (I)
wherein
Z denotes a residue obtained by removing hydroxyl groups from a compound having 3 to 9 hydroxyl groups;
AO denotes an oxyalkylene group having 3 to 4 carbon atoms;
EO denotes an oxyethylene group;
BO denotes an oxyalkylene group having 4 carbon atoms;
a denotes 3 to 9;
l, m, and n denote the average addition mole numbers of AO, EO and BO, respectively, and 1≤l≤50, 1≤m≤50 and 0.5≤n≤5;
the weight ratio of AO to EO (AO/EO) ranges from 1/5 to 5/1; and
AO and EO may have been added randomly or in the form of blocks; and
(d) water,
wherein
the amount of the (a) first compound(s) in the composition is 1% by weight or more, preferably 3% by weight or more, and more preferably 5% by weight or more, relative to the total weight of the composition.

The (a) first compound may be in the form of a lactone.

The (a) first compound may be gluconolactone.

The amount of the (a) first compound(s) in the composition according to the present invention may be from 1% to 20% by weight, preferably from 3% to 15% by weight, and more preferably from 5% to 10% by weight, relative to the total weight of the composition.

The (b) second compound may be niacinamide.

The amount of the (b) second compound(s) in the composition according to the present invention may be from 1% to 20% by weight, preferably from 2% to 15% by weight, and more preferably from 3% to 10% by weight, relative to the total weight of the composition.

The (c) alkylene oxide derivative may be represented by the following chemical formula (II):

Gly-{O(PO)ₛ(EO)ₜ-(BO)ᵤH}₃ (II)
wherein
Gly denotes a residue obtained by removing hydroxyl groups from glycerin;
PO denotes an oxypropylene group;
EO denotes an oxyethylene group;
s and t denote the average addition mole numbers of PO and EO, respectively, and have a value ranging from 1 to 50;
the weight ratio of PO to EO (PO/EO) ranges from 1/5 to 5/1;
BO denotes an oxyalkylene group having 4 carbon atoms; and
u denotes the average addition mole number of BO, and ranges from 0.5 to 5.

The (c) alkylene oxide derivative may be PEG/PPG/Polybutylene glycol-8/5/3 glycerin.

The amount of the (c) alkyleneoxide derivative(s) in the composition according to the present invention may be from 0.1% to 12% by weight, preferably from 0.5% to 10% by weight, and more preferably from 1% to 8% by weight, relative to the total weight of the composition.

The composition according to the present invention may further comprise (e) at least one oil in an amount of 1% by weight or less, preferably 0.1% by weight or less, and more preferably 0.01% by weight or less, relative to the total weight of the composition.

The composition according to the present invention may further comprise (f) at least one additional compound selected from alpha-hydroxy acids, beta-hydroxy acids, phytic acid, and mixtures thereof, which is different from the (a) first compound.

The amount of the (f) additional compound(s) in the composition according to the present invention may be from 0.001% to 5% by weight, preferably from 0.01% to 4% by weight, and more preferably from 0.05% to 3% by weight, relative to the total weight of the composition.

The composition according to the present invention may be a cosmetic composition, preferably a skin cosmetic composition, and more preferably a skin peeling composition.

The present invention also relates to a cosmetic process for treating a keratin substance, preferably for treating the skin, and more preferably for peeling the skin, comprising the step of applying the composition according to the present invention to the keratin substance.

The present invention also relates to a use of a combination of
(b) at least one second compound selected from Vitamin B3 and derivatives thereof; and
(c) at least one alkyleneoxide derivative represented by the following chemical formula (I):

Z—{O(AO)ₗ(EO)ₘ-(BO)ₙH}ₐ (I)
wherein
Z denotes a residue obtained by removing hydroxyl groups from a compound having 3 to 9 hydroxyl groups;
AO denotes an oxyalkylene group having 3 to 4 carbon atoms;
EO denotes an oxyethylene group;
BO denotes an oxyalkylene group having 4 carbon atoms;
a denotes 3 to 9;
l, m, and n denote the average addition mole numbers of AO, EO and BO, respectively, and 1≤l≤50, 1≤m≤50 and 0.5≤n≤5;
the weight ratio of AO to EO (AO/EO) ranges from 1/5 to 5/1; and
AO and EO may have been added randomly or in the form of blocks;
   in a composition comprising:
(a) at least one first compound selected from polyhydroxy acids and salts thereof; and
(d) water
wherein
the amount of the (a) first compound(s) in the composition is 1% by weight or more, preferably 3% by weight or more, and more preferably 5% by weight or more, relative to the total weight of the composition,
in order to reduce stickiness and skin discomfort such as skin irritation caused by the composition when the composition is applied onto the skin.

### BEST MODE FOR CARRYING OUT THE INVENTION

After diligent research, the inventors have discovered that it is possible to provide a transparent composition which can provide less stickiness and the reduction of skin discomfort such as skin irritation, when the composition is applied onto the skin, even if the composition includes a relatively large amount of polyhydroxy acid or salt thereof.

Thus, one aspect of the present invention is a composition comprising:
(a) at least one first compound selected from polyhydroxy acids and salts thereof;
(b) at least one second compound selected from Vitamin B3 and derivatives thereof;
(c) at least one alkyleneoxide derivative represented by the following chemical formula (I):

Z—{O(AO)ₗ(EO)ₘ-(BO)ₙH}ₐ (I)
wherein
Z denotes a residue obtained by removing hydroxyl groups from a compound having 3 to 9 hydroxyl groups;
AO denotes an oxyalkylene group having 3 to 4 carbon atoms;
EO denotes an oxyethylene group;
BO denotes an oxyalkylene group having 4 carbon atoms;
a denotes 3 to 9;
l, m, and n denote the average addition mole numbers of AO, EO and BO, respectively, and 1≤l≤50, 1≤m≤50 and 0.5≤n≤5;
the weight ratio of AO to EO (AO/EO) ranges from 1/5 to 5/1; and
AO and EO may have been added randomly or in the form of blocks; and
(d) water,
wherein
the amount of the (a) first compound(s) in the composition is 1% by weight or more, preferably 3% by weight or more, and more preferably 5% by weight or more, relative to the total weight of the composition.

The composition according to the present invention is transparent.

The composition according to the present invention is less sticky, although the composition includes a relatively large amount of polyhydroxy acid or salt thereof, i.e., 1% by weight or more, preferably 3% by weight or more, and more preferably 5% by weight or more, relative to the total weight of the composition, and therefore, it can provide a reduced sticky feeling to the touch.

The term “sticky” here means a property which provides a tacky feeling to the skin.

Thus, the composition according to the present invention can provide an excellent feeling during use, in particular, excellent feeling of the skin after application of the composition.

The composition according to the present can provide less skin discomfort. Skin discomfort here means unpleasantness which does not relate to texture such as stickiness. Examples of unpleasantness include tingling, soreness, burning, and the like.

The scope of skin discomfort includes skin irritation. Thus, the composition according to the present invention can be less irritating to the skin. In other words, the composition according to the present invention can be mild to the skin.

The composition according to the present invention can provide reduced risk of skin inflammation.

Accordingly, the composition according to the present invention is transparent, and can provide less stickiness and reduced skin discomfort such as reduced skin irritation, when the composition is applied onto the skin, although the composition includes a relatively large amount of polyhydroxy acid or salt thereof.

The composition according to the present invention is useful for peeling off the stratum corneum or horny layer from the skin.

The composition according to the present invention can provide cosmetic effects of exfoliating the skin, improving the condition of the skin, and the like, in terms of both the appearance and the feel to the touch of the skin, including smoothing the skin, refining and brightening the skin tone, reducing blemishes, pores and blackheads.

Hereinafter, the composition according to the present invention will be explained in a more detailed manner.

### Polyhydroxy Acid

The composition according to the present invention comprises (a) at least one first compound selected from polyhydroxy acids and salts thereof. If two or more first compounds are used, they may be the same or different.

The term “polyhydroxy acid” here means an organic compound which has at least one carboxyl group and a plurality of hydroxy groups. The number of carboxyl groups in the polyhydroxy acid is not limited, but one or two carboxyl groups are preferable, and one carboxyl group is more preferable. The number of hydroxyl groups in the polyhydroxy acid is also not limited, but 2 to 10 are preferable, and 2 to 6 are more preferable. The number of carbon atoms in the polyhydroxy acid is not limited, but 3 to 11 are preferable, and 3 to 8 are more preferable.

The above organic compound may be aliphatic or aromatic. In other words, the polyhydroxy acid may be selected from aliphatic polyhydroxy acids or aromatic polyhydroxy acids. It is preferable that the aliphatic polyhydroxy acid be selected from sugar acids.

Examples of the polyhydroxy acid include, dihydroxy propanoic acid such as glyceric acid; trihydroxy butanoic acid such as erythronic acid and threonic acid; tetrahydroxy pentanoic acid such as ribonic acid, arabinoic acid, xylonic acid, and lyxonic acid; pentahydroxy hexanoic acid such as allonic acid, altronic acid, gluconic acid, mannoic acid, gulonic acid, idonic acid, galactonic acid, and talonic acid; hexahydroxy heptanoic acid such as glucoheptanoic acid, galactoheptonic acid; tartaric acid; lactobionic acid, maltobionic acid, and mixtures thereof.

It is preferable that the (a) first compound be in the form of a lactone. The lactone ring of the (a) first compound in the form of a lactone may be saturated. Examples of the (a) first compound include, gluconolactone, ribonolactone, and mixtures thereof.

It is more preferable that the (a) first compound be gluconolactone.

The type of the salt is not limited. Examples of the salt include alkaline metal salts such as sodium salt and potassium salt; alkaline earth metal salts such as calcium salt and magnesium salt; zinc salts; iron salts; ammonium salts; amine salts such as monoethanolamine salt, diethanolamine salt and triethanolamine salt; and mixtures thereof. Sodium salt is preferable. If two or more salts are used, they may be the same or different.

The amount of the (a) first compound(s) in the composition according to the present invention is 1% by weight or more, preferably 3% by weight or more, and more preferably 5% by weight or more, relative to the total weight of the composition.

On the other hand, the amount of the (a) first compound(s) in the composition according to the present invention may be 20% by weight or less, preferably 15% by weight or less, and more preferably 10% by weight or less, relative to the total weight of the composition.

The amount of the (a) first compound(s) in the composition according to the present invention may be from 1% to 20% by weight, preferably from 3% to 15% by weight, and more preferably from 5% to 10% by weight, relative to the total weight of the composition.

### Vitamin B3 and Derivatives Thereof

The composition according to the present invention comprises (b) at least one second compound selected from Vitamin B3 and derivatives thereof. If two or more second compounds are used, they may be the same or different.

Vitamin B3, also called Vitamin PP, is a compound of the following formula:

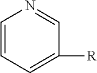

in which R may be —CONH₂ (niacinamide), —COOH (nicotinic acid or niacin), or CH₂OH (nicotinyl alcohol), —CO—NH—CH₂—COOH (nicotinuric acid) or —CO—NH—OH (niconityl hydroxamic acid). Niacinamide is preferable.

Vitamin B3 derivatives that may be mentioned include, for example, nicotinic acid esters such as tocopherol nicotinate, amides derived from niacinamide by substitution of the hydrogen groups of —CONH₂, products from reaction with carboxylic acids and amino acids, esters of nicotinyl alcohol and of carboxylic acids such as acetic acid, salicylic acid, glycolic acid or palmitic acid.

Mention may also be made of the following derivatives: 2-chloronicotinamide, 6-methylnicotinamide, 6-aminonicotinamide, N-methylnicotinamide, N,N-dimethylnicotinamide, N-(hydroxymethyl) nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine, methyl nicotinate and sodium nicotinate.

Other Vitamin B3 derivatives that may also be mentioned include its inorganic salts, such as chlorides, bromides, iodides or carbonates, and its organic salts, such as the salts obtained by reaction with carboxylic acids, such as acetate, salicylate, glycolate, lactate, malate, citrate, mandelate, tartrate, etc.

It is preferable that the (b) second compound selected from Vitamin B3 and derivatives thereof have a log P of from −0.7 to 6, and preferably from −0.5 to 4.

A log P value is a value for the base-ten logarithm of the apparent octan-1-ol/water partition coefficient. The log P values are known and are determined by a standard test which determines the concentration of the (b) second compound in octan-1-ol and water. The log P may be calculated according to the method described in the article by Meylan and Howard: Atom/Fragment contribution method for estimating octanol-water partition coefficients, J. Pharm. Sci., 84: 83-92, 1995. This value may also be calculated using numerous commercially available software packages, which determine the log P as a function of the structure of a molecule. By way of example, mention may be made of the Epiwin software from the United States Environmental Agency.

The values may especially be calculated using the ACD (Advanced Chemistry Development) Solaris software V4.67; they may also be obtained from Exploring QSAR: hydrophobic, electronic and steric constants (ACS professional reference book, 1995). There is also an Internet site which provides estimated values (address: http://esc.syrres.com/interkow/kowdemo.htm).

It is more preferable that the (b) second compound be niacinamide.

The amount of the (b) second compound(s) in the composition according to the present invention may be 1% by weight or more, preferably 2% by weight or more, and more preferably 3% by weight or more, relative to the total weight of the composition.

On the other hand, the amount of the (b) second compound(s) in the composition according to the present invention may be 20% by weight or less, preferably 15% by weight or less, and more preferably 10% by weight or less, relative to the total weight of the composition.

The amount of the (b) second compound(s) in the composition according to the present invention may be from 1% to 20% by weight, preferably from 2% to 15% by weight, and more preferably from 3% to 10% by weight, relative to the total weight of the composition.

### Alkyleneoxide Derivative

The composition according to the present invention comprises (c) at least one alkyleneoxide derivative. If two or more alkyleneoxide derivatives are used, they may be the same or different.

The (c) alkyleneoxide derivative is represented by the following chemical formula (1):

Z—{O(AO)ₗ(EO)ₘ-(BO)ₙH}ₐ (I)
wherein
Z denotes a residue obtained by removing hydroxyl groups from a compound having 3 to 9 hydroxyl groups;
AO denotes an oxyalkylene group having 3 to 4 carbon atoms;
EO denotes an oxyethylene group;
BO denotes an oxyalkylene group having 4 carbon atoms;
a denotes 3 to 9;
l, m, and n denote the average addition mole numbers of AO, EO and BO, respectively, and 1≤l≤50, 1≤m≤50 and 0.5≤n≤5;
the weight ratio of AO to EO (AO/EO) ranges from 1/5 to 5/1; and
AO and EO may have been added randomly or in the form of blocks.

In the alkylene oxide derivative represented by formula (I), Z denotes a residue obtained by removing hydroxyl groups from a compound having 3 to 9 hydroxyl groups, and a denotes the number of hydroxyl groups of the compound and denotes 3 to 9. As examples of compounds having 3 to 9 hydroxyl groups, mention may be made of, for example, in the case of a=3, glycerin, and trimethylolpropane; in the case of a=4, erythritol, pentaerythritol, sorbitol, alkylglycosides, and diglycerin; in the case of a=5, xylitol; in the case of a=6, dipentaerythritol, sorbitol, and inositol; in the case of a=8, sucrose, and trehalose; in the case of a=9, maltitol; mixtures thereof; and the like. Preferably, Z denotes a residue obtained by removing hydroxyl group(s) from a compound having 3 to 6 hydroxyl groups, and a satisfies 3≤a≤6. As the compound having 3 to 9 hydroxyl groups, glycerin or trimethylolpropane is preferable, and in particular, glycerin is preferable. In the case of a≤2, poor compatibility with oil components such as fats and oils is exhibited, and blending stability in an oil-based formulation tends to be impaired. In the case of 10≤a, stickiness is caused.

AO denotes an oxyalkylene group having 3 to 4 carbon atoms. As examples thereof, mention may be made of, for example, an oxypropylene group, an oxybutylene group (an oxy-n-butylene group, an oxyisobutylene group, or an oxy-t-butylene group), an oxytrimethylene group, an oxytetramethylene group, and the like. The oxypropylene group and oxybutylene group are preferable, and the oxypropylene group is more preferable.

l denotes the average addition mole number of AO, and satisfies 1≤l≤50, and preferably 2≤l≤20. m denotes the average addition mole number of EO, and satisfies 1≤m≤50, and preferably 2≤m≤20. If l is 0, stickiness occurs. On the other hand, if l exceeds 50, moisturizing effects are decreased. In addition, if m is 0, moisturizing effects are decreased. On the other hand, if m exceeds 50, stickiness occurs.

The weight ratio of AO to EO (AO/EO) ranges from 1/5 to 5/1, and preferably ranges from 1/4 to 4/1. If AO/EO is below 1/5, stickiness occurs. On the other hand, if AO/EO exceeds 5/1, the moisturizing sensation is decreased. The order of adding AO and EO is not particularly specified. AO and EO may have been added randomly or in the form of blocks. In order to obtain superior effects of preventing skin roughness, AO and EO preferably have been added randomly.

BO denotes an oxyalkylene group having 4 carbon atoms. As examples thereof, mention may be made of, for example, an oxybutylene group (an oxy-n-butylene group, an oxy-isobutylene group, or an oxy-t-butylene group), an oxytetramethylene group, and the like. The oxybutylene group is preferable.

n denotes the average addition mole number of BO, and satisfies 0.5<n≤5, preferably 0.8≤n≤3, and more preferably 1≤n≤3. If n is below 0.5, stickiness occurs. On the other hand, if n exceeds 5, moisturizing effects are decreased. In formula (I), it is necessary that (BO)ₙ bond to the terminal hydrogen atom.

The alkylene oxide derivatives represented by formula (I) can be produced by means of known methods. For example, the alkylene oxide derivatives represented by formula (I) can be obtained by additive-polymerizing ethylene oxide and an alkylene oxide having 3 to 4 carbon atoms to a compound having 3 to 9 hydroxyl groups, and subsequently reacting with an alkylene oxide having 4 carbon atoms. When additive-polymerizing ethylene oxide and an alkylene oxide having 3 to 4 carbon atoms to a compound having 3 to 9 hydroxyl groups, the ethylene oxide and alkylene oxide may be polymerized randomly or in the form of blocks.

Among the alkylene oxide derivatives represented by formula (I), preferable examples of the aforementioned alkylene oxide derivatives include, for example, an alkylene oxide derivative (polyoxybutylene polyoxyethylene polyoxypropylene glycerol) represented by formula (II) shown below:

Gly-{O(PO)ₛ(EO)ₜ-(BO)ᵤH}₃ (II)
wherein
Gly denotes a residue obtained by removing hydroxyl groups from glycerin;
PO denotes an oxypropylene group;
EO denotes an oxyethylene group;
s and t denote the average addition mole numbers of PO and EO, respectively, and have a value ranging from 1 to 50;
the weight ratio of PO to EO (PO/EO) ranges from 1/5 to 5/1;
BO denotes an oxyalkylene group having 4 carbon atoms; and
u denotes the average addition mole number of BO, and ranges from 0.5 to 5.

The aforementioned alkylene oxide derivative represented by formula (II) can be obtained by adding propylene oxide and ethylene oxide to glycerin, in the ratio of 3 to 150 mole equivalents of each of propylene oxide and ethylene oxide with respect to glycerin, and subsequently, adding the alkylene oxide having 4 carbon atoms in the ratio of 1.5 to 15 mole equivalents thereof with respect to glycerin.

In the case of adding the aforementioned alkylene oxides to glycerin, the addition reactions are carried out with an alkali catalyst, a phase transfer catalyst, a Lewis acid catalyst, or the like. In general, an alkali catalyst such as potassium hydroxide is preferably employed.

Among the alkylene oxide derivatives represented by formula (I), more preferable derivatives are obtained by adding 6 to 10 mol of ethylene oxide and 3 to 7 mol of propylene oxide to glycerin, and subsequently, adding 2 to 4 mol of butylene oxide.

Among the alkylene oxide derivatives represented by formula (I), a further more preferable derivative is polyoxybutylene polyoxyethylene polyoxypropylene glycerol, which is obtained by adding 8 mol of ethylene oxide and 5 mol of propylene oxide to glycerin, and subsequently, adding 3 mol of butylene oxide, and which has an INCI name of PEG/PPG/polybutylene glycol-8/5/3 glycerin. PEG/PPG/polybutylene glycol-8/5/3 glycerin is commercially available as the trade name WILBRIDE S-753 from NOF Corporation.

The amount of the (c) alkyleneoxide derivative(s) in the composition according to the present invention may be 0.1% by weight or more, preferably 0.5% by weight or more, and more preferably 1% by weight or more, relative to the total weight of the composition.

On the other hand, the amount of the (c) alkyleneoxide derivative(s) in the composition according to the present invention may be 12% by weight or less, preferably 10% by weight or less, and more preferably 8% by weight or less, relative to the total weight of the composition.

The amount of the (c) alkyleneoxide derivative(s) in the composition according to the present invention may be from 0.1% to 12% by weight, preferably from 0.5% to 10% by weight, and more preferably from 1% to 8% by weight, relative to the total weight of the composition.

### Water

The composition according to the present invention comprises (d) water.

The amount of the (d) water in the composition according to the present invention may be 40% by weight or more, preferably 50% by weight or more, and more preferably 60% by weight or more, relative to the total weight of the composition.

On the other hand, the amount of the (d) water in the composition according to the present invention may be 90% by weight or less, preferably 80% by weight or less, and more preferably 70% by weight or less, relative to the total weight of the composition.

The amount of the (d) water in the composition according to the present invention may be from 40% to 90% by weight, preferably from 50% to 80% by weight, and more preferably from 60% to 70% by weight, relative to the total weight of the composition.

### Oil

The composition according to the present invention may comprise (e) at least one oil in a limited amount. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The (e) oil may be a non-polar oil such as a hydrocarbon oil, a silicone oil, or the like; a polar oil such as a plant or animal oil and an ester oil or an ether oil; or a mixture thereof.

The (e) oil may be selected from the group consisting of oils of plant or animal origin, synthetic oils, silicone oils, hydrocarbon oils, and fatty alcohols.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, grape seed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

Esters of C₄-C₂₂ dicarboxylic or tricarboxylic acids and of C₁-C₂₂ alcohols, and esters of monocarboxylic, dicarboxylic, or tricarboxylic acids and of non-sugar C₄-C₂₆ dihydroxy, trihydroxy, tetrahydroxy, or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides, or polysaccharides.

Examples of suitable sugars that may be mentioned include sucrose (or saccharose), glucose, galactose, ribose, fucose, maltose, fructose, mannose, arabinose, xylose, and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, linoleates, linolenates, caprates, and arachidonates, or mixtures thereof such as, especially, oleopalmitate, oleostearate, and palmitostearate mixed esters, as well as pentaerythrityl tetraethyl hexanoate.

More particularly, use is made of monoesters and diesters and especially sucrose, glucose, or methylglucose monooleates or dioleates, stearates, behenates, oleopalmitates, linoleates, linolenates, and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate), and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of the formula:

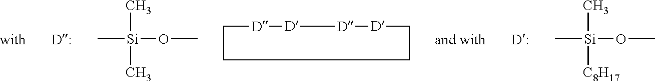

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

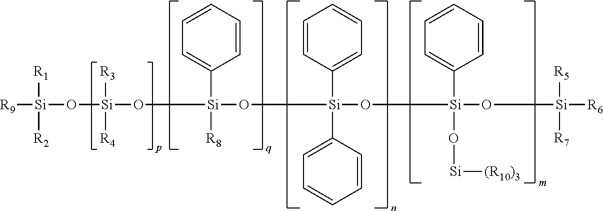

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl, or butyl radicals, and
m, n, p, and q are, independently of each other, integers of 0 to 900 inclusive, preferably 0 to 500 inclusive, and more preferably 0 to 100 inclusive,
with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250, and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

The hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane, and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R-OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from cetyl alcohol, octyldodecanol, hexyldecanol, and mixtures thereof.

It is also preferable that the (e) oil be chosen from oils with a molecular weight below 600 g/mol.

Preferably, the (e) oil has a low molecular weight such as below 600 g/mol, chosen among ester oils with a short hydrocarbon chain or chains (C₁-C₁₂) (e.g., isopropyl lauroyl sarcosinate, isopropyl myristate, isopropyl palmitate, isononyl isononanoate, and ethyl hexyl palmitate), silicone oils (e.g., volatile silicones such as cyclohexasiloxane), hydrocarbon oils (e.g., isododecane, isohexadecane, and squalane), branched and/or unsaturated fatty alcohol (C₁₂-C₃₀) type oils such as octyldodecanol and oleyl alcohol, and ether oils such as dicaprylyl ether.

The (e) oil may be chosen from polar oils, preferably from ester oils, and more preferably isopropyl myristate, and from non-polar oils, preferably ether oils, and more preferably dicaprylyl ether.

It is preferable that the amount of the (e) oil(s) in the composition according to the present invention be 1% by weight or less, more preferably 0.1% by weight or less, and even more preferably 0.01% by weight or less, relative to the total weight of the composition.

It is most preferable that the composition according to the present invention comprise no oil.

However, the amount of the (e) oil(s) in the composition according to the present invention may be 0.001% by weight or more.

### Additional Compound

The composition according to the present invention may comprise (f) at least one additional compound selected from alpha-hydroxy acids, beta-hydroxy acids, phytic acid, and mixtures thereof, which is different from the (a) first compound. If two or more additional compounds are used, they may be the same or different.

The term “alpha-hydroxy acid”, or “AHA”, here means a carboxylic acid which has at least one carboxyl group and at least one hydroxyl group separated by one carbon atom. Thus, in other words, AHA is a carboxylic acid which has at least one hydroxyl group on the adjacent (alpha) carbon atom.

The alpha-hydroxy acid may be selected from, for example glycolic acid, lactic acid, malic acid, citric acid, mandelic acid, and mixtures thereof, preferably from glycolic acid, citric acid, and mixtures thereof, more preferably from glycolic acid.

The term “beta-hydroxy acid”, or “BHA”, here means a carboxylic acid which has at least one carboxyl group and at least one hydroxyl group separated by two carbon atoms.

The beta-hydroxy acid may be salicylic acid.

It is preferable to use, as the (f) additional compounds, a combination of at least one alpha-hydroxy acid and at least one beta-hydroxy acid, and more preferably a combination of glycolic acid and salicylic acid, with phytic acid.

It is known that AHA activates the protease in the stratum corneum to enhance the degradation of corneodesmosomes, while in the dermis layer, the AHA can promote collagen formation, which subsequently leads to desquamation of skin. In addition, it has been reported that AHA also decreases facial pores and blackheads, most likely by targeting enzyme activation and protein functions linked to sebum production.

It is known that BHA adds keratolytic activity which promotes the peeling and shedding of cells in the stratum corneum layer. It also decongests the skin by softening the sebum which blocks the pores. For this reason, it is effective for improving desquamation and especially effective for acne and oily skin types as well.

The amount of the (f) additional compound(s) in the composition according to the present invention may be 0.001% by weight or more, preferably 0.01% by weight or more, and more preferably 0.05% by weight or more, relative to the total weight of the composition.

On the other hand, the amount of the (f) additional compound(s) in the composition according to the present invention may be 5% by weight or less, preferably 4% by weight or less, and more preferably 3% by weight or less, relative to the total weight of the composition.

The amount of the (f) additional compound(s) in the composition according to the present invention may be from 0.001% to 5% by weight, preferably from 0.01% to 4% by weight, and more preferably from 0.05% to 3% by weight, relative to the total weight of the composition.

### Hyaluronic Acid Ingredient

The composition according to the present invention may comprise at least one hyaluronic acid ingredient selected from the group consisting of hyaluronic acid, hyaluronic acid salts, hyaluronic acid derivatives, hyaluronic acid derivative salts, and mixtures thereof. A single type of hyaluronic acid ingredient may be used, but two or more different types of hyaluronic acid ingredients may be used in combination.

Hyaluronic acid is a predominant glucosaminoglycan found in the skin. Thus, the fibroblasts synthesize predominantly collagens, matrix glycoproteins other than collagens (fibronectin, laminin), proteoglycans and elastin. The keratinocytes, for their part, synthesize predominantly sulfated glycosaminoglycans and hyaluronic acid. Hyaluronic acid is also called hyaluronan.

Hyaluronic acid is present in the free state in the epidermis and in the dermis and is responsible for turgescence of the skin. This polysaccharide can in fact retain a large volume of water, corresponding to up to 1000 times its weight. In this sense, hyaluronic acid plays an important role in increasing the amounts of water bound in the tissue, and also in the mechanical properties of the skin and in wrinkle formation.

Hyaluronic acid can be represented by the following chemical formula.

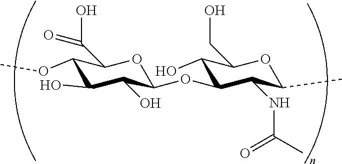

In the context of the present invention, the term “hyaluronic acid” covers in particular the basic unit of hyaluronic acid of formula:

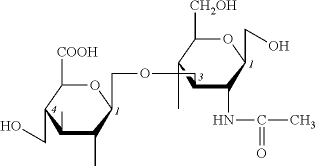

It is the smallest fraction of hyaluronic acid comprising a disaccharide dimer, namely D-glucuronic acid and N-acetylglucosamine.

Hyaluronic acid derivatives may be selected from the group consisting of hydrolyzed hyaluronic acid, acetylated hyaluronic acid, cationic hyaluronic acid, and mixtures thereof.

The cationic hyaluronic acid comprises at least one cationic moiety. The cationic moiety may be a trialkyl ammonium group such as —N⁺(CH₃)₃. The cationic moiety may include at least one hydroxyl group. The examples of the cationic group include —CH₂—CH(OH)—CH₂—N⁺(CH₃)₃.

Examples of the cationic hyaluronic acid includes hydroxypropyl trimonium hyaluronate.

As hyaluronic acid salts or hyaluronic acid derivative salts, mention may be made of alkaline metal salts such as sodium salt and potassium salt, alkaline earth metal salts such as magnesium salts, ammonium salts, and mixtures thereof.

The molecular weight of the hyaluronic acid ingredient is not limited. The molecular weight of the hyaluronic acid ingredient may be 5 kDa or more, preferably 20 kDa or more, and more preferably 100 kDa or more. The molecular weight of the hyaluronic acid ingredient may be 20 MDa or less, preferably 10 MDa or less, and more preferably 2,000 kDa or less. Thus, the molecular weight of the hyaluronic acid ingredient may be from 5 kDa to 20 MDa, preferably from 20 kDa to 10 MDa, and more preferably from 100 kDa to 2,000 kDa.

Unless otherwise defined in the descriptions, “molecular weight” may mean a number average molecular weight.

The hyaluronic acid ingredient may in particular be hyaluronic acid supplied by the company Hyactive under the trade name CPN (MW: 10 to 150 kDa), by the company Soliance under the trade name Cristalhyal (MW: 1 to 1.4 MDa), by the company Bioland under the name Nutra HA (MW: 820 000 Da), by the company Bioland under the name Nutra AF (MW: 69 000 Da), or by the company Bioland under the name Oligo HA (MW: 6100 Da).

A single hyaluronic acid ingredient with a single molecular weight, or a combination of two or more hyaluronic acid ingredients with different molecular weights may be used.

The amount of the hyaluronic acid ingredient(s) in the composition according to the present invention may be 0.0001% by weight or more, preferably 0.0005% by weight or more, and more preferably 0.001% by weight or more, relative to the total weight of the composition.

The amount of the hyaluronic acid ingredient(s) in the composition according to the present invention may be 1% by weight or less, preferably 0.5% by weight or less, and more preferably 0.1% by weight or less, relative to the total weight of the composition.

Thus, the amount of the hyaluronic acid ingredient(s) in the composition according to the present invention may range from 0.0001% to 1% by weight, preferably from 0.0005% to 0.5% by weight, and more preferably from 0.001% to 0.1% by weight, relative to the total weight of the composition.

### Polyol

The composition according to the present invention may comprise at least one polyol. If two or more polyols are used, they may be the same or different.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

Polyols used in the present invention are liquid at ambient temperature such as 25°° C. under atmospheric pressure (760 mmHg or 105 Pa).

The polyol may be a C₂₋₂₄ polyol, preferably a C₂₋₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof. The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, C₆-C₂₄ polyethyleneglycol, 1,3-propanediol, 1,4-butanediol, and 1,5-pentanediol.

The amount of the polyol(s) in the composition used in the present invention may be 1% by weight or more, preferably 3% by weight or more, and more preferably 5% by weight or more, relative to the total weight of the composition.

On the other hand, the amount of the polyol(s) in the composition used in the present invention may be 20% by weight or less, preferably 15% by weight or less, and more preferably 10% by weight or less, relative to the total weight of the composition.

The amount of the polyol(s) in the composition used in the present invention may range from 1% to 20% by weight, preferably from 3% to 15% by weight, and more preferably from 5% to 10% by weight, relative to the total weight of the composition.

### Nonionic Surfactant

The composition according to the present invention may comprise at least one nonionic surfactant. If two or more nonionic surfactants are used, they may be the same or different.

The nonionic surfactant may have an HLB (Hydrophilic Lipophilic Balance) value of from 8.0 to 14.0, preferably from 9.0 to 13.5, and more preferably from 10.0 to 13.0. If two or more nonionic surfactants are used, the HLB value is determined by the weight average of the HLB values of all the nonionic surfactants.

The nonionic surfactant may be chosen from:
(1) surfactants chosen from polyglyceryl fatty acid esters, polyoxyalkylenated alkyl glycerides, and polyoxyalkylenated fatty ethers;
(2) mixed esters of fatty acid or of fatty alcohol, of carboxylic acid and of glycerol;
(3) fatty acid esters of sugars and fatty alcohol ethers of sugars;
(4) surfactants chosen from fatty esters of sorbitan and oxyalkylenated fatty esters of sorbitan, and oxyalkylenated fatty esters;
(5) block copolymers of ethylene oxide (A) and of propylene oxide (B),
(6) polyoxyethylenated (1-40 EO) and polyoxypropylenated (1-30 PO) alkyl (C₁₆-C₃₀) ethers,
(7) silicone surfactants, and
(8) mixtures thereof.

The surfactant (1) may be a fluid at a temperature of less than or equal to 45° C.

The surfactant (1) may be in particular:
polyglyceryl fatty acid esters of at least one, preferably one, fatty acid comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2-12 glycerols, preferably 2-10 glycerols and more preferably 2-8 glycerols;
polyoxyethylenated (PEGylated) alkyl glycerides such as polyethylene glycol derivatives of a mixture of mono-, di- and tri-glycerides of caprylic and capric acids (preferably 2 to 30 ethylene oxide units, more preferably 2 to 20 ethylene oxide units, and even more preferably 2 to 10 ethylene oxide units), e.g., PEG-6 Caprylic/Capric Glycerides, PEG-7 Caprylic/Capric Glycerides, and PEG-7 glyceryl cocoate;
polyoxyethylenated fatty ethers of at least one, preferably one, fatty alcohol comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2 to 60 ethylene oxides, preferably from 2 to 30 ethylene oxides, and more preferably from 2 to 10 ethylene oxides; and mixtures thereof.

It is preferable that the polyglyceryl fatty acid ester have a polyglycerol moiety derived from 2 to 10 glycerols, more preferably from 2 to 8 glycerols, and further more preferably 4 to 6 glycerols.

The polyglyceryl fatty acid ester may be chosen from the mono, di and tri esters of saturated or unsaturated acid, preferably saturated acid, including 8 to 22 carbon atoms, preferably 8 to 18 carbon atoms, and more preferably 8 to 12 carbon atoms, such as caprylic acid, capric acid, lauric acid, oleic acid, stearic acid, isostearic acid, and myristic acid.

The polyglyceryl fatty acid ester may be selected from the group consisting of PG2 caprate, PG2 dicaprate, PG2 tricaprate, PG2 caprylate, PG2 dicaprylate, PG2 tricaprylate, PG2 laurate, PG2 dilaurate, PG2 trilaurate, PG2 myristate, PG2 dimyristate, PG2 trimyristate, PG2 stearate, PG2 distearate, PG2 tristearate, PG2 isostearate, PG2 diisostearate, PG2 triisostearate, PG2 oleate, PG2 dioleate, PG2 trioleate, PG3 caprate, PG3 dicaprate, PG3 tricaprate, PG3 caprylate, PG3 dicaprylate, PG3 tricaprylate, PG3 laurate, PG3 dilaurate, PG3 trilaurate, PG3 myristate, PG3 dimyristate, PG3 trimyristate, PG3 stearate, PG3 distearate, PG3 tristearate, PG3 isostearate, PG3 diisostearate, PG3 triisostearate, PG3 oleate, PG3 dioleate, PG3 trioleate, PG4 caprate, PG4 dicaprate, PG4 tricaprate, PG4 caprylate, PG4 dicaprylate, PG4 tricaprylate, PG4 laurate, PG4 dilaurate, PG4 trilaurate, PG4 myristate, PG4 dimyristate, PG4 trimyristate, PG4 stearate, PG4 distearate, PG4 tristearate, PG4 isostearate, PG4 diisostearate, PG4 triisostearate, PG4 oleate, PG4 dioleate, PG4 trioleate, PG5 caprate, PG5 dicaprate, PG5 tricaprate, PG5 caprylate, PG5 dicaprylate, PG5 tricaprylate, PG5 laurate, PGS dilaurate, PG5 trilaurate, PG5 myristate, PG5 dimyristate, PG5 trimyristate, PG5 stearate, PG5 distearate, PG5 tristearate, PG5 isostearate, PG5 diisostearate, PG5 triisostearate, PG5 oleate, PG5 dioleate, PG5 trioleate, PG6 caprate, PG6 dicaprate, PG6 tricaprate, PG6 caprylate, PG6 dicaprylate, PG6 tricaprylate, PG6 laurate, PG6 dilaurate, PG6 trilaurate, PG6 myristate, PG6 dimyristate, PG6 trimyristate, PG6 stearate, PG6 distearate, PG6 tristearate, PG6 isostearate, PG6 diisostearate, PG6 triisostearate, PG6 oleate, PG6 dioleate, PG6 trioleate, PG10 caprate, PG10 dicaprate, PG10 tricaprate, PG10 caprylate, PG10 dicaprylate, PG10 tricaprylate, PG10 laurate, PG10 dilaurate, PG10 trilaurate, PG10 myristate, PG10 dimyristate, PG10 trimyristate, PG10 stearate, PG10 distearate, PG10 tristearate, PG10 isostearate, PG10 diisostearate, PG10 triisostearate, PG10 oleate, PG10 dioleate, and PG10 trioleate.

The polyoxyalkylenated fatty ethers, preferably polyoxyethylenated fatty ethers, may comprise from 2 to 60 ethylene oxide units, preferably from 2 to 30 ethylene oxide units, and more preferably from 2 to 10 ethylene oxide units. The fatty chain of the ethers may be chosen in particular from lauryl, behenyl, arachidyl, stearyl and cetyl units, and mixtures thereof, such as cetearyl. Examples of ethoxylated fatty ethers which may be mentioned are lauryl alcohol ethers comprising 2, 3, 4, and 5 ethylene oxide units (CTFA names: Laureth-2, Laureth-3, Laureth-4, and Laureth-5, such as the products sold under the names Nikkol BL-2 by the company Nikko Chemicals, Emalex 703 by the company Nihon Emulsion Co., Ltd, Nikkol BL-4 by the company Nikko Chemicals, and EMALEX 705 by the company Nihon Emulsion Co., Ltd.

The (2) mixed esters of fatty acid, or of fatty alcohol, of carboxylic acid and of glycerol, which can be used as the above nonionic surfactant, may be chosen in particular from the group comprising mixed esters of fatty acid or of fatty alcohol with an alkyl or alkenyl chain containing from 8 to 22 carbon atoms, preferably from 8 to 18 carbon atoms, and more preferably from 8 to 12 carbon atoms, and of α-hydroxy acid and/or of succinic acid, with glycerol. The α-hydroxy acid may be, for example, citric acid, lactic acid, glycolic acid or malic acid, and mixtures thereof.

The alkyl chain of the fatty acids or alcohols from which are derived the mixed esters which can be used in the present invention may be linear or branched, and saturated or unsaturated. They may especially be stearate, isostearate, linoleate, oleate, behenate, arachidonate, palmitate, myristate, laurate, caprate, isostearyl, stearyl, linoleyl, oleyl, behenyl, myristyl, lauryl or capryl chains, and mixtures thereof.

As examples of mixed esters which can be used in the present invention, mention may be made of the mixed ester of glycerol and of the mixture of citric acid, lactic acid, linoleic acid and oleic acid (CTFA name: Glyceryl citrate/lactate/linoleate/oleate) sold by the company Hüls under the name Imwitor 375; the mixed ester of succinic acid and of isostearyl alcohol with glycerol (CTFA name: Isostearyl diglyceryl succinate) sold by the company Hüls under the name Imwitor 780 K; the mixed ester of citric acid and of stearic acid with glycerol (CTFA name: Glyceryl stearate citrate) sold by the company Hüls under the name Imwitor 370; the mixed ester of lactic acid and of stearic acid with glycerol (CTFA name: Glyceryl stearate lactate) sold by the company Danisco under the name Lactodan B30 or Rylo LA30.

The (3) fatty acid esters of sugars, which can be used as the above nonionic surfactant, may be chosen in particular from the group comprising esters or mixtures of esters of C₈-C₂₂ fatty acid and of sucrose, of maltose, of glucose or of fructose, and esters or mixtures of esters of C₁₄-C₂₂ fatty acid and of methylglucose.

The C₈-C₂₂ or C₁₄-C₂₂ fatty acids forming the fatty unit of the esters which can be used in the present invention comprise a saturated or unsaturated linear alkyl or alkenyl chain containing, respectively, from 8 to 22 or from 14 to 22 carbon atoms. The fatty unit of the esters may be chosen in particular from stearates, behenates, arachidonates, palmitates, myristates, laurates and caprates, and mixtures thereof. Stearates are preferably used.

As examples of esters or mixtures of esters of fatty acid and of sucrose, of maltose, of glucose or of fructose, mention may be made of sucrose monostearate, sucrose distearate and sucrose tristearate and mixtures thereof, such as the products sold by the company Croda under the name Crodesta F50, F70, F110 and F160; and examples of esters or mixtures of esters of fatty acid and of methylglucose which may be mentioned are methylglucose polyglyceryl-3 distearate, sold by the company Goldschmidt under the name Tego-care 450. Mention may also be made of glucose or maltose monoesters such as methyl o-hexadecanoyl-6-D-glucoside and o-hexadecanoyl-6-D-maltoside.

The (3) fatty alcohol ethers of sugars, which can be used as the above nonionic surfactant, may be solid at a temperature of less than or equal to 45° C. and may be chosen in particular from the group comprising ethers or mixtures of ethers of C₈-C₂₂ fatty alcohol and of glucose, of maltose, of sucrose or of fructose, and ethers or mixtures of ethers of a C₁₄-C₂₂ fatty alcohol and of methylglucose. These are in particular alkylpolyglucosides.

The C₈-C₂₂ or C₁₄-C₂₂ fatty alcohols forming the fatty unit of the ethers which may be used in the present invention comprise a saturated or unsaturated, linear alkyl or alkenyl chain containing, respectively, from 8 to 22 or from 14 to 22 carbon atoms. The fatty unit of the ethers may be chosen in particular from decyl, cetyl, behenyl, arachidyl, stearyl, palmityl, myristyl, lauryl, capryl and hexadecanoyl units, and mixtures thereof, such as cetearyl.

As examples of fatty alcohol ethers of sugars, mention may be made of alkylpolyglucosides such as decylglucoside and laurylglucoside, which is sold, for example, by the company Henkel under the respective names Plantaren 2000 and Plantaren 1200, cetostearyl glucoside optionally as a mixture with cetostearyl alcohol, sold for example, under the name Montanov 68 by the company SEPPIC, under the name Tego-care CG90 by the company Goldschmidt and under the name Emulgade KE3302 by the company Henkel, as well as arachidyl glucoside, for example in the form of a mixture of arachidyl alcohol and behenyl alcohol and arachidyl glucoside, sold under the name Montanov 202 by the company SEPPIC.

The surfactant used more particularly is sucrose monostearate, sucrose distearate or sucrose tristearate and mixtures thereof, methylglucose polyglyceryl-3 distearate and alkylpolyglucosides.

The (4) fatty esters of sorbitan and oxyalkylenated fatty esters of sorbitan which may be used as the above nonionic surfactant may be chosen from the group comprising C₁₆-C₂₂ fatty acid esters of sorbitan and oxyethylenated C₁₆-C₂₂ fatty acid esters of sorbitan. They may be formed from at least one fatty acid comprising at least one saturated linear alkyl chain containing, respectively, from 16 to 22 carbon atoms, and from sorbitol or from ethoxylated sorbitol. The oxyethylenated esters may generally comprise from 1 to 100 ethylene glycol units and preferably from 2 to 40 ethylene oxide (EO) units.

These esters may be chosen in particular from stearates, behenates, arachidates, palmitates, and mixtures thereof. Stearates and palmitates are preferably used.

As examples of the above nonionic surfactant that can be used in the present invention, mention may be made of sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65.

The (4) oxyalkylenated fatty esters, preferably ethoxylated fatty esters, which may be used as the above nonionic surfactant, may be esters formed from 1 to 100 ethylene oxide units, preferably from 2 to 60 ethylene oxide units, and more preferably from 2 to 30 ethylene oxide units, and from at least one fatty acid chain containing from 8 to 22 carbon atoms, preferably from 8 to 18 carbon atoms, and more preferably from 8 to 12 carbon atoms. The fatty chain in the esters may be chosen in particular from stearate, behenate, arachidate and palmitate units, and mixtures thereof. Examples of ethoxylated fatty esters which may be mentioned are the ester of stearic acid comprising 40 ethylene oxide units, such as the product sold under the name Myrj 52 (CTFA name: PEG-40 stearate) by the company ICI, as well as the ester of behenic acid comprising 8 ethylene oxide units (CTFA name: PEG-8 behenate), such as the product sold under the name Compritol HD5 ATO by the company Gattefosse.

The (5) block copolymers of ethylene oxide (A) and of propylene oxide (B), which may be used as the above nonionic surfactant, may be chosen in particular from block copolymers of formula (I):

HO(C₂H₄O)ₓ(C₃H₆O)_{y}(C₂H₄O)₂H (I)

in which x, y and z are integers such that x+z ranges from 2 to 100 and y ranges from 14 to 60, and mixtures thereof, and more particularly from the block copolymers of formula (I) having an HLB value ranging from 8.0 to 14.0.

The (6) polyoxyethylenated (1-40 EO) and polyoxypropylenated (1-30 PO) alkyl (C₁₆-C₃₀) ethers, which may be used as the above nonionic surfactant, may be selected from the group consisting of:
PPG-6 Decyltetradeceth-30; Polyoxyethlene (30) Polyoxypropylene (6) Tetradecyl Ether such as those sold as Nikkol PEN-4630 from Nikko Chemicals Co.,
PPG-6 Decyltetradeceth-12; Polyoxyethylene (12) Polyoxypropylene (6) Tetradecyl Ether such as those sold as Nikkol PEN-4612 from Nikko Chemicals Co.,
PPG-13 Decyltetradeceth-24; Polyoxyethylene (24) Polyoxypropylene (13) Decyltetradecyl Ether such as those sold as UNILUBE 50MT-2200B from NOF Corporation,
PPG-6 Decyltetradeceth-20; Polyoxyethylene (20) Polyoxypropylene (6) Decyltetradecyl Ether such as those sold as Nikkol PEN-4620 from Nikko Chemicals Co.,
PPG-4 Ceteth-1; Polyoxyethylene (1) Polyoxypropylene (4) Cetyl Ether such as those sold as Nikkol PBC-31 from Nikko Chemicals Co.,
PPG-8 Ceteth-1; Polyoxyethylene (1) Polyoxypropylene (8) Cetyl Ether such as those sold as Nikkol PBC-41 from Nikko Chemicals Co.,
PPG-4 Ceteth-10; Polyoxyethylene (10) Polyoxypropylene (4) Cetyl Ether such as those sold as Nikkol PBC-33 from Nikko Chemicals Co.,
PPG-4 Ceteth-20; Polyoxyethylene (20) Polyoxypropylene (4) Cetyl Ether such as those sold as Nikkol PBC-34 from Nikko Chemicals Co.,
PPG-5 Ceteth-20; Polyoxyethylene (20) Polyoxypropylene (5) Cetyl Ether such as those sold as Procetyl AWS from Croda Inc.,
PPG-8 Ceteth-20; Polyoxyethylene (20) Polyoxypropylene (8) Cetyl Ether such as those sold as Nikkol PBC-44 from Nikko Chemicals Co., and
PPG-23 Steareth-34; Polyoxyethylene Polyoxypropylene Stearyl Ether (34 EO) (23 PO) such as those sold as Unisafe 34S-23 from Pola Chemical Industries. They can provide a composition with stability for a long time, even though the temperature of the composition is increased and decreased in a relatively short period of time.

It may be more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-6 Decyltetradeceth-20, PPG-5 Ceteth-20, PPG-8 Ceteth-20, and PPG-23 Steareth-34.

It may be even more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-5 Ceteth-20, and PPG-8 Ceteth-20.

As (7) silicone surfactants, which may be used as the above nonionic surfactant, mention may be made of those disclosed in documents U.S. Pat. Nos. 5,364,633 and 5,411,744.

The (7) silicone surfactant as the above nonionic surfactant may preferably be a compound of formula (I):

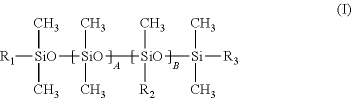

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; with the proviso that A and B are not simultaneously equal to zero;
x is an integer ranging from 1 to 6;
y is an integer ranging from 1 to 30;
z is an integer ranging from 0 to 5.

According to one preferred embodiment of the present invention, in the compound of formula (I), the alkyl radical is a methyl radical, x is an integer ranging from 2 to 6 and y is an integer ranging from 4 to 30.

As examples of silicone surfactants of formula (I), mention may be made of the compounds of formula (II):

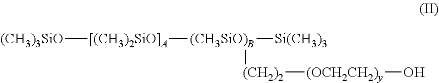

in which A is an integer ranging from 20 to 105, B is an integer ranging from 2 to 10 and y is an integer ranging from 10 to 20.

As examples of silicone surfactants of formula (I), mention may also be made of the compounds of formula (III):

H—(OCH₂CH₂)_{y}—(CH₂)₃—[(CH₃)₂SiO]_{A′}—(CH₂)₃—(OCH₂CH₂)_{y}—OH (III)

in which A′ and y are integers ranging from 10 to 20.

Compounds of the present invention which may be used are those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; A is 27, B is 3 and y is 12.

The compound Q4-3667 is a compound of formula (III) in which A is 15 and y is 13.

The amount of the nonionic surfactant(s) in the composition used in the present invention may be 0.01% by weight or more, preferably 0.05% by weight or more, and more preferably 0.1% by weight or more, relative to the total weight of the composition.

On the other hand, the amount of the nonionic surfactant(s) in the composition used in the present invention may be 5% by weight or less, preferably 3% by weight or less, and more preferably 1% by weight or less, relative to the total weight of the composition.

The amount of the nonionic surfactant(s) in the composition used in the present invention may range from 0.01% to 5% by weight, preferably from 0.05% to 3% by weight, and more preferably from 0.1% to 1% by weight, relative to the total weight of the composition.

### Other Ingredients

The composition according to the present invention may comprise at least one monoalcohol which is in the form of a liquid at room temperature (25° C.), such as a linear or branched monoalcohol comprising from 1 to 6 carbon atoms, for example, ethanol, propanol, butanol, isopropanol, isobutanol, pentanol, and hexanol.

The amount of the monoalcohol(s) in the composition according to the present invention may be 0.01% by weight or more, preferably 0.1% by weight or more, and more preferably 1% by weight or more, relative to the total weight of the composition.

On the other hand, the amount of the monoalcohol(s) in the composition according to the present invention may be 15% by weight or less, preferably 10% by weight or less, and more preferably 5% by weight or less, relative to the total weight of the composition.

Thus, the amount of the monoalcohol(s) in the composition according to the present invention may range from 0.01% to 15% by weight, preferably from 0.1% to 10% by weight, and more preferably from 1% to 5% by weight, relative to the total weight of the composition.

The composition according to the present invention may contain at least one pH adjusting agent.

As the pH adjusting agent, at least one acidifying agent and/or at least one basifying agent (alkaline agent) may be used.

The acidifying agents can be, for example, inorganic or organic acids, for instance hydrochloric acid, phosphoric acid, sulfonic acid, and carboxylic acids such as lactic acid.

The acidifying agent may be present in an amount ranging from less than 5% by weight, preferably from 3% by weight or less, and more preferably from 1% by weight or less, relative to the total weight of the composition.

The basifying agent or alkaline agent can be, for example, any inorganic or organic basic agents which are commonly used in cosmetic products such as ammonia; alkanolamines such as mono-, di- and tri-ethanolamine, isopropanolamine; metal hydroxide such as alkaline metal hydroxide (e.g., sodium and potassium hydroxides); urea, guanidine and their derivatives; and diamines such as those described in the structure below:

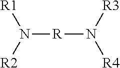

wherein
R denotes an alkylene such as propylene optionally substituted by a hydroxyl or a C₁-C₄ alkyl radical, and R₁, R₂, R₃, and R₄ independently denote a hydrogen atom, an alkyl radical, or a C₁-C₄ hydroxyalkyl radical, which may be exemplified by 1,3-propanediamine, and derivatives thereof. Alkaline metal hydroxide such as potassium hydroxide is preferable.

The pH of the composition according to the present invention may be less than 7, preferably 6 or less, and more preferably 5.5 or less. The pH of the composition according to the present invention may be 3.5 or more, preferably 4.0 or more, and more preferably 4.5 or more. Thus, the pH of the composition according to the present invention may be from 3.5 to less than 7, preferably from 4.0 to 6, and more preferably from 4.5 to 5.5.

The composition according to the present invention may also include various adjuvants conventionally used in cosmetic compositions, such as thickeners, anionic, non-ionic, cationic, and amphoteric or zwitterionic polymers, anionic, cationic, and amphoteric surfactants, antioxidants, coloring agents, chelating agents, sequestering agents, fragrances, dispersing agents, conditioning agents, film-forming agents, preservatives, co-preservatives, and mixtures thereof, except for the ingredients as explained above.

### Preparation

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

### Form

The composition according to the present invention is transparent.

The transparency may be measured by measuring the turbidity (for example, turbidity can be measured with a 2100Q (marketed by Hach Company) having a round cell (25 mm in diameter and 60 mm height) and a tungsten filament lamp which can emit visible light (between 400 and 800 nm, preferably from 400 to 500 nm). The measurement can be performed on the undiluted composition. The blank may be determined with distilled water.

The composition according to the present invention may have a turbidity of 50 NTU or less, preferably 40 NTU or less, more preferably 30 NTU or less, more preferably 20 NTU or less, more preferably 10 NTU or less, and even more preferably 5 NTU or less.

The form of the composition according to the present invention is not limited. Thus, the composition according to the present invention may be in the form of a solution, a gel, or an emulsion.

However, it is preferable that the composition according to the present invention not be in the form of an emulsion.

Nevertheless, if the composition according to the present invention is in the form of an emulsion, the composition according to the present invention comprises a continuous phase and dispersed phases. The dispersed phases are dispersed in the continuous phase. Thus, the dispersed phase may be in the form of a particle. The continuous phase can comprise (d) water. The dispersed phase may comprise oil if present.

The composition according to the present invention may be in the form of a nano- or micro-emulsion, if the composition comprises oil.

The “micro-emulsion” may be defined in two ways, namely, in a broader sense and in a narrower sense. That is to say, there are one case (“microemulsion in the narrow sense”) in which the microemulsion refers to a thermodynamically stable isotropic single liquid phase containing a ternary system having three ingredients of an oily component, an aqueous component and a surfactant, and another case (“micro-emulsion in the broad sense”) in which, among thermodynamically unstable typical emulsion systems, the microemulsion additionally includes those such emulsions presenting transparent or translucent appearances due to their smaller particle sizes (Satoshi Tomomasa, et al., Oil Chemistry, Vol. 37, No. 11 (1988), pp. 48-53). “Micro-emulsion” as used herein refers to a “micro-emulsion in the narrow sense,” i.e., a thermodynamically stable isotropic single liquid phase.

The micro-emulsion refers to either one state of an O/W (oil-in-water) type microemulsion in which oil is solubilized by micelles, a W/O (water-in-oil) type microemulsion in which water is solubilized by reverse micelles, or a bicontinuous microemulsion in which the number of associations of surfactant molecules are rendered infinite so that both the aqueous phase and oil phase have a continuous structure.

The micro-emulsion may have a dispersed phase with a particle size of 100 nm or less, preferably 80 nm or less, and more preferably 60 nm or less, measured by laser granulometry.

“Nano-emulsion” here means an emulsion characterized by a dispersed phase with a size of less than 350 nm, the dispersed phase being stabilized by a crown of surfactant(s) if present that may optionally form a liquid crystal phase of lamellar type, at the dispersed phase/continuous phase interface. In the absence of specific opacifiers, the transparency of the nano-emulsion arises from the small size of the dispersed phase, this small size being obtained by virtue of the use of mechanical energy and especially a high-pressure homogenizer.

Nano-emulsions can be distinguished from micro-emulsions by their structure. Specifically, micro-emulsions are thermodynamically stable dispersions formed from, for example, micelles which are formed by surfactant(s) if present and swollen with oil. Furthermore, micro-emulsions do not require substantial mechanical energy in order to be prepared.

The nano-emulsion may have a dispersed phase with a particle size of 300 nm or less, preferably 200 nm or less, and more preferably 100 nm or less, measured by laser granulometry.

The composition according to the present invention may be in the form of an O/W nano- or micro-emulsion, or a W/O nano- or micro-emulsion. It is preferable that the composition according to the present invention be in the form of an O/W nano- or micro-emulsion.

The O/W architecture or structure, which consists of oil phases dispersed in an aqueous phase, has an external aqueous phase, and therefore if the composition according to the present invention has the O/W architecture or structure, it can provide a pleasant feeling during use because of the feeling of immediate freshness that the aqueous phase can provide.

The particle size of the oil if present may be 100 nm or less, preferably 75 nm or less, and more preferably 50 nm or less. The particle size can be measured by a dynamic light scattering method. The particle size measurement can be performed by, for example, the Particle Size Analyzer ELSZ-2000 series, marketed by Otsuka Electronics Co., Ltd.

The particle size can be a volume-average particle diameter or a number-average particle diameter, preferably a volume-average particle diameter.

### Process and Use

It is preferable that the composition according to the present invention be a cosmetic composition, preferably a skin cosmetic composition, and more preferably a skin peeling composition. The skin peeling here means peeling the superficial surface of the skin, in particular the horny layer of the skin.

The skin here encompasses facial skin, neck skin, and scalp. The composition according to the present invention may also be used for mucosae such as lips, and the like.

The composition according to the present invention can be used for a non-therapeutic process, such as a cosmetic process, for treating a keratin substance, preferably for treating the skin, and more preferably for peeling the skin, by being applied to the keratin substance.

Thus, the present invention also relates to a cosmetic process for treating a keratin substance, preferably for treating the skin, and more preferably for peeling the skin, comprising the step of applying the composition according to the present invention to the keratin substance in particular the skin.

The present invention may also relate to a use of the composition according to the present invention as a cosmetic product or in a cosmetic product such as skin care products. In other words, the composition according to the present invention can be used, as it is, as a cosmetic product. Alternatively, the composition according to the present invention can be used as an element of a cosmetic product. For example the composition according to the present invention can be added to or combined with any other elements to form a cosmetic product.

The care product may be in the form of a solution, a gel, a lotion, and the like.

As the composition according to the present invention includes at least one first compound selected from polyhydroxy acids and salts thereof, the composition according to the present invention can be used for skin peeling while providing less stickiness and reduced skin discomfort such as reduced skin irritation.

Another aspect of the present invention may relate to a use of a combination of:
(b) at least one second compound selected from Vitamin B3 and derivatives thereof; and
(c) at least one alkyleneoxide derivative represented by the following chemical formula (I):

Z—{O(AO)ₗ(EO)ₘ-(BO)ₙH}ₐ (1)
wherein
Z denotes a residue obtained by removing hydroxyl groups from a compound having 3 to 9 hydroxyl groups;
AO denotes an oxyalkylene group having 3 to 4 carbon atoms;
EO denotes an oxyethylene group;
BO denotes an oxyalkylene group having 4 carbon atoms;
a denotes 3 to 9;
l, m, and n denote the average addition mole numbers of AO, EO and BO, respectively, and 1≤l≤50, 1≤m≤50 and 0.5≤n≤5;
the weight ratio of AO to EO (AO/EO) ranges from 1/5 to 5/1; and
AO and EO may have been added randomly or in the form of blocks;
   in a composition comprising:
(a) at least one first compound selected from polyhydroxy acids and salts thereof; and
(d) water
wherein
the amount of the (a) first compound(s) in the composition is 1% by weight or more, preferably 3% by weight or more, and more preferably 5% by weight or more, relative to the total weight of the composition,
in order to reduce stickiness and skin discomfort such as skin irritation caused by the composition when the composition is applied onto the skin.

The above explanations regarding the ingredients (a) to (d), as well as the optional ingredients, for the composition according to the present invention can apply to those for the above use according to the present invention. The explanations regarding the preparation and forms of the composition according to the present invention can also apply to those of the composition described in the above use according to the present invention.

### EXAMPLES

The present invention will be described in more detail by way of examples which however should not be construed as limiting the scope of the present invention.

### Example 1 and Comparative Examples 1-3

The following compositions according to Example 1 and Comparative Examples 1-3, shown in Table 1, were prepared by mixing the components shown in Table 1. The numerical values for the amounts of the components shown in Table 1 are all based on “% by weight” as raw materials.

TABLE 1

| | | Comp. | Comp. | Comp. |
|---|---|---|---|---|
| Ingredients | Ex. 1 | Ex. 1 | Ex. 2 | Ex. 3 |
| Gluconolactone | 10 | 10 | 10 | 10 |
| Niacinamide | 5 | — | 5 | — |
| PEG/PPG/Polybutylene | 4 | 4 | — | — |
| Glycol-8/5/3 Glycerin | | | | |
| Water | qsp 100 | qsp 100 | qsp 100 | qsp 100 |
| Methyl Gluceth-20 | 5 | 5 | 5 | 5 |
| Ethanol | 3 | 3 | 3 | 3 |
| Butylene Glycol | 5 | 5 | 5 | 5 |
| Pentylene Glycol | 3 | 3 | 3 | 3 |
| Hydroxyethyl Cellulose | 0.2 | 0.2 | 0.2 | 0.2 |
| Sodium Hyaluronate | 0.001 | 0.001 | 0.001 | 0.001 |
| Chlorphenesine | 0.2 | 0.2 | 0.2 | 0.2 |
| Potassium Hydroxide | qs pH 5 | qs pH 5 | qs pH 5 | qs pH 5 |
| Salicylic Acid | 0.2 | 0.2 | 0.2 | 0.2 |
| Phytic Acid | 0.2 | 0.2 | 0.2 | 0.2 |
| Glycolic Acid | 0.1 | 0.1 | 0.1 | 0.1 |
| Fagus Sylvatica Bud Extract | 2 | 2 | 2 | 2 |
| PPG-6 Decyltetradeceth-30 | 0.3 | 0.3 | 0.3 | 0.3 |
| Fragrance | 0.05 | 0.05 | 0.05 | 0.05 |
| Vitis Vinifera (Grape) Seed Oil | 0.01 | 0.01 | 0.01 | 0.01 |
| Transparency | Very | Very | Very | Very |
| | Good | Good | Good | Good |
| Non-Stickiness | Good | Very | Very | Very |
| | | Good | Poor | Poor |
| Skin Irritation | Good | Poor | NA | NA |

NA: Not Available (due to stickiness)

### [Evaluations]

### (Transparency)

The transparency of the compositions according to Example 1 and Comparative Examples 1-3 was also determined by visual observation in accordance with the following criteria.
Very Good: Transparent
Good: Slightly Translucent
Poor: Translucent
Very Poor: Opaque

The results are shown in Table 1.

### (Non-Stickiness)

The stickiness of the compositions according to Example 1 and Comparative Examples 1-3 was measured by using a Texture Analyzer (TA. XT plus by Eco Instruments Co., Ltd.).

10 mg of each of the compositions according to Example 1 and Comparative Examples 1-3 was applied on a bioskin (square shape: 2 cm×2 cm) available from Beaulax. A metal head probe of the Texture Analyzer above the bioskin was moved down to touch the bioskin with a determined force. The metal head was then lifted up while measuring negative force data. The negative force data indicate how much force is required when lifting up the metal head probe, and relate to stickiness. The peak of the negative force data was recorded. The lower the negative force data is, the higher the stickiness is. This was repeated 100 times for each composition, and the value of the peak negative force was averaged. The absolute values of the averaged peak negative force for the compositions according to Example 1 and Comparative Examples 1-3 are categorized in accordance with the following criteria.
Very Good: ≤100
Good: 100< and ≤110
Poor: 110< and ≤120
Very Poor: 120<

The results are shown in Table 1.

### (Skin Irritation)

In vitro evaluation using EpiSkin™ was performed.

EpiSkin™ tissues (20ER34, 20ER35) and maintenance medium were obtained from Shanghai EPISKIN Biotechnology Co., Ltd with required QC reports. They were received and subjected to recovery in an incubator at 37° C. and 5% CO₂ for at least 24 hours before conducting experiments.

A preliminary test on MTT interaction was performed for all the compositions according to Example 1 and Comparative Examples 1-3. Each of the compositions was incubated with 3.3 mg/mL MTT solution at 37° C. for at least 15 minutes and any color change was recorded for further testing.

For cytokine analysis, a topical application of 35 μL of each composition for 48 hours of treatment was performed on two replicate skin tissues in one experiment for individual concentrations. At the end of treatment, basal media were collected and sent out for analysis using luminex systems. EpiSkin™ tissues were collected for viability. Final data was corrected for viability and the average levels were expressed as pg/mL for individual cytokines. At least two folds of changes will be considered for ranking analysis.

The grading was categorized by the following two criteria based on the cytokine level.
Good: IL-8<2 and IL-1α<2.8
Poor: IL-8>2 or IL-1α>2.8

Good means no risk of skin irritation

Poor means risk of skin irritation

The results are shown in Table 1.

### Example 2 and Comparative Examples 4-6

The following compositions according to Example 2 and Comparative Examples 4-6, shown in Table 1, were prepared by mixing the components shown in Table 2. The numerical values for the amounts of the components shown in Table 2 are all based on “% by weight” as raw materials.

TABLE 2

| | | Comp. | Comp. | Comp. |
|---|---|---|---|---|
| Ingredients | Ex. 2 | Ex. 4 | Ex. 5 | Ex. 6 |
| Gluconolactone | 5 | 5 | 5 | 5 |
| Niacinamide | 5 | — | 5 | — |
| PEG/PPG/Polybutylene | 4 | 4 | — | — |
| Glycol-8/5/3 Glycerin | | | | |
| Water | qsp 100 | qsp 100 | qsp 100 | qsp 100 |
| Methyl Gluceth-20 | 5 | 5 | 5 | 5 |
| Ethanol | 3 | 3 | 3 | 3 |
| Butylene Glycol | 5 | 5 | 5 | 5 |
| Pentylene Glycol | 3 | 3 | 3 | 3 |
| Hydroxyethyl Cellulose | 0.2 | 0.2 | 0.2 | 0.2 |
| Sodium Hyaluronate | 0.001 | 0.001 | 0.001 | 0.001 |
| Chlorphenesine | 0.2 | 0.2 | 0.2 | 0.2 |
| Potassium Hydroxide | qs pH 5 | qs pH 5 | qs pH 5 | qs pH 5 |
| Salicylic Acid | 0.2 | 0.2 | 0.2 | 0.2 |
| Phytic Acid | 0.2 | 0.2 | 0.2 | 0.2 |
| Glycolic Acid | 0.1 | 0.1 | 0.1 | 0.1 |
| Fagus Sylvatica Bud Extract | 2 | 2 | 2 | 2 |
| PPG-6 Decyltetradeceth-30 | 0.3 | 0.3 | 0.3 | 0.3 |
| Fragrance | 0.05 | 0.05 | 0.05 | 0.05 |
| Vitis Vinifera (Grape) Seed Oil | 0.01 | 0.01 | 0.01 | 0.01 |
| Transparency | Very | Very | Very | Very |
| | Good | Good | Good | Good |
| Non-Stickiness | Very | Good | Very | Very |
| | Good | | Poor | Poor |
| Skin Irritation | Good | Poor | NA | NA |

NA: Not Available (due to stickiness)

### [Evaluations]

### (Transparency)

The transparency of the compositions according to Example 2 and Comparative Examples 4-6 was also determined by visual observation in accordance with the following criteria.
Very Good: Transparent
Good: Slightly Translucent
Poor: Translucent
Very Poor: Opaque

The results are shown in Table 2.

### (Non-Stickiness)

The stickiness of the compositions according to Example 2 and Comparative Examples 4-6 was measured by using a Texture Analyzer (TA. XT plus by Eco Instruments Co., Ltd.).

10 mg of each of the compositions according to Example 2 and Comparative Examples 4-6 was applied on a bioskin (square shape: 2 cm×2 cm) available from Beaulax. A metal head probe of the Texture Analyzer above the bioskin was moved down to touch the bioskin with a determined force. The metal head was then lifted up while measuring negative force data. The negative force data indicate how much force is required when lifting up the metal head probe, and relate to stickiness. The peak of the negative force data was recorded. The lower the negative force data is, the higher the stickiness is. This was repeated 100 times for each composition, and the value of the peak negative force was averaged. The absolute values of the averaged peak negative force for the compositions according to Example 2 and Comparative Examples 4-6 are categorized in accordance with the following criteria.
Very Good: ≤100
Good: 100< and ≤110
Poor: 110< and ≤120
Very Poor: 120<

The results are shown in Table 2.

### (Skin Irritation)

In vitro evaluation using EpiSkin™ was performed.

EpiSkin™ tissues (20ER34, 20ER35) and maintenance medium were obtained from Shanghai EPISKIN Biotechnology Co., Ltd with required QC reports. They were received and subjected to recovery in an incubator at 37° C. and 5% CO₂ for at least 24 hours before conducting experiments.

A preliminary test on MTT interaction was performed for all the compositions according to Example 2 and Comparative Examples 4-6. Each of the compositions was incubated with 3.3 mg/mL MTT solution at 37° C. for at least 15 minutes and any color change was recorded for further testing.

For cytokine analysis, a topical application of 35 μL of each composition for 48 hours of treatment was performed on two replicate skin tissues in one experiment for individual concentrations. At the end of treatment, basal media were collected and sent out for analysis using luminex systems. EpiSkin™ tissues were collected for viability. Final data was corrected for viability and the average levels were expressed as pg/mL for individual cytokines. At least two folds of changes will be considered for ranking analysis.

The grading was categorized by the following two criteria based on the cytokine level.
Good: IL-8<2 and IL-1α<2.8
Poor: IL-8>2 or IL-1α>2.8

Good means no risk of skin irritation

Poor means risk of skin irritation

The results are shown in Table 2.

1. A transparent composition, comprising:
(a) at least one first compound selected from polyhydroxy acids and salts thereof;
(b) at least one second compound selected from Vitamin B3 and derivatives thereof;
(c) at least one alkyleneoxide derivative represented by chemical formula (I):
Z—{O(AO)ₗ(EO)ₘ-(BO)ₙH}ₐ (I)
wherein
Z denotes a residue obtained by removing hydroxyl groups from a compound having 3 to 9 hydroxyl groups;
AO denotes an oxyalkylene group having 3 to 4 carbon atoms;
EO denotes an oxyethylene group;
BO denotes an oxyalkylene group having 4 carbon atoms;
a denotes 3 to 9;
l, m, and n denote average addition mole numbers of AO, EO and BO, respectively, and 1≤l≤50, 1≤m≤50 and 0.5≤n≤5;
a weight ratio of AO to EO (AO/EO) ranges from 1/5 to 5/1; and
AO and EO may have been added randomly or in a form of blocks; and
(d) water,
wherein
an amount of the (a) first compound(s) in the composition is 1% by weight or more, relative to a total weight of the composition.

2. The composition according to claim 1, wherein the (a) first compound is in a form of a lactone.

3. The composition according to claim 1, wherein the (a) first compound is gluconolactone.

4. The composition according to claim 1, wherein the amount of the (a) first compound(s) in the composition is from 1% to 20% by weight, relative to the total weight of the composition.

5. The composition according to claim 1, wherein the (b) second compound is niacinamide.

6. The composition according to claim 1, wherein an amount of the (b) second compound(s) in the composition is from 1% to 20% by weight, relative to the total weight of the composition.

7. The composition according to claim 1, wherein the (c) alkylene oxide derivative is represented by chemical formula (II):
Gly-{O(PO)ₛ(EO)ₜ-(BO)ᵤH}₃ (II)
wherein
Gly denotes a residue obtained by removing hydroxyl groups from glycerin;
PO denotes an oxypropylene group;
EO denotes an oxyethylene group;
s and t denote average addition mole numbers of
PO and EO, respectively, and have a value ranging from 1 to 50;
a weight ratio of PO to EO (PO/EO) ranges from 1/5 to 5/1;
BO denotes an oxyalkylene group having 4 carbon atoms; and
u denotes an average addition mole number of BO, and ranges from 0.5 to 5.

8. The composition according to claim 1, wherein the (c) alkylene oxide derivative is PEG/PPG/Polybutylene glycol-8/5/3 glycerin.

9. The composition according to claim 1, wherein an amount of the (c) alkyleneoxide derivative(s) in the composition is from 0.1% to 12% by weight, relative to the total weight of the composition.

10. The composition according to claim 1, wherein the composition further comprises (e) at least one oil in an amount of 1% by weight or less, relative to the total weight of the composition.

11. The composition according to claim 1, wherein the composition further comprises (f) at least one additional compound selected from alpha-hydroxy acids, beta-hydroxy acids, phytic acid, and mixtures thereof, which is different from the (a) first compound.

12. The composition according to claim 11, wherein an amount of the (f) additional compound(s) in the composition is from 0.001% to 5% by weight, relative to the total weight of the composition.

13. The composition according to claim 1, wherein the composition is a cosmetic composition.

14. A cosmetic process for treating a keratin substance, comprising:
applying the composition according to claim 1 to the keratin substance.

15. A process comprising:
reducing stickiness and skin discomfort caused by a composition when the composition is applied onto a skin by including a combination of
(b) at least one second compound selected from Vitamin B3 and derivatives thereof; and
(c) at least one alkyleneoxide derivative represented by chemical formula (I):
Z—{O(AO)ₗ(EO)ₘ-(BO)ₙH}ₐ (I)
wherein
Z denotes a residue obtained by removing hydroxyl groups from a compound having 3 to 9 hydroxyl groups;
AO denotes an oxyalkylene group having 3 to 4 carbon atoms;
EO denotes an oxyethylene group;
BO denotes an oxyalkylene group having 4 carbon atoms;
a denotes 3 to 9;
l, m, and n denote average addition mole numbers of AO, EO and BO, respectively, and 1≤l≤50, 1≤m≤50 and 0.5≤n≤5;
a weight ratio of AO to EO (AO/EO) ranges from 1/5 to 5/1; and
AO and EO may have been added randomly or in a form of blocks;
in the composition comprising:
(a) at least one first compound selected from polyhydroxy acids and salts thereof; and
(d) water
wherein
an amount of the (a) first compound(s) in the composition is 1% by weight or more, relative to a total weight of the composition.
